(19) United States
(12) Patent Application Publication
(10) Pub. No.: US 2005/0222130 A1
(43) Pub. Date: Oct. 6, 2005

(54) Inhibitors of RhoA kinase production/activation for relaxing the features of and/or for decontracting the skin

(75) Inventors: Dominique Fagot, Paris (FR); Pascal Portes, Nogent Sur Marne (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, Paris (FR)

(21) Appl. No.: 11/097,311

(22) Filed: Apr. 4, 2005

Related U.S. Application Data
(60) Provisional application No. 60/559,969, filed on Apr. 7, 2004.

(30) Foreign Application Priority Data
Apr. 2, 2004 (FR) 04/50658

(51) Int. Cl. A61K 31/551; A61K 31/44

(52) U.S. Cl. 514/218; 514/357

(57) ABSTRACT

Inhibitors of RhoA kinase production and/or activation are suited for relaxing the features and/or for decontracting the skin and thus are useful for preventing and/or combating the signs of aging of the skin, in particular wrinkles and notably expression wrinkles.

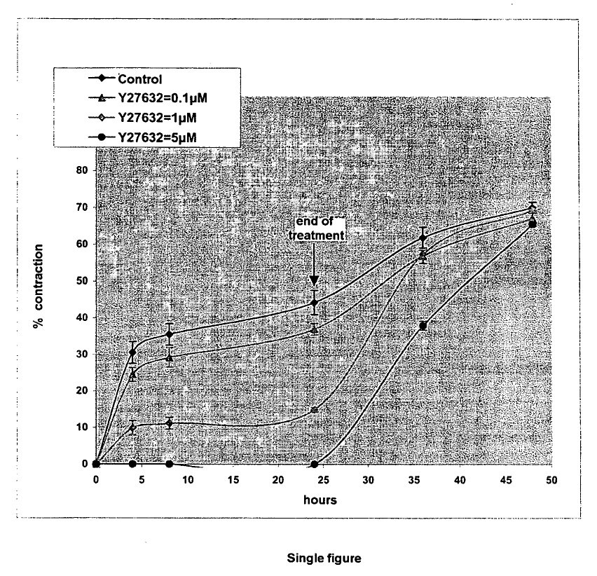

### CROSS-REFERENCE TO PRIORITY/PROVISIONAL APPLICATIONS

This application claims priority under 35 U.S.C. § 119 of FR 04/50658, filed Apr. 2, 2004, and of provisional application Ser. No. 60/559,969, filed Apr. 7, 2004, each hereby expressly incorporated by reference and each assigned to the assignee hereof. This application is also a continuation of said '969 provisional.

### BACKGROUND OF THE INVENTION

1. Technical Field of the Invention

The invention relates to the formulation, into cosmetic compositions, of an effective amount of at least one inhibitor of RhoA kinase production and/or activation, as an agent for relaxing the features and/or decontracting the skin.

In particular, the said inhibitor is an inhibitor of RhoA kinase-dependent RhoA activation.

The invention also relates to cosmetic compositions for topical application, comprising at least one inhibitor of RhoA kinase production and/or activation, and also to a cosmetic process (regime/regimen) for treating the skin, especially aged and/or wrinkled skin.

2. Description of Background and/or Related and/or Prior Art

Women, and even men, currently have a tendency to wish to look youthful for as long as possible and consequently seek to fade out the age marks on the skin, which are reflected in particular by wrinkles and fine lines. In this respect, the media and the fashion world report about products intended to keep the skin radiant and wrinkle-free for as long as possible, which are signs of youthful skin, and all the more so since the physical appearance acts on the psyche and/or on the morale.

It is known that wrinkles are not all the same and that their origins are different: some are due to age-related morphological or physiological changes, while others are due to “aggravating” factors such as the movements of the face, exposure to sunlight or hormonal changes.

Facial wrinkles thus vary with age and also with the number and intensity of the aggravating factors.

Hitherto, wrinkles and fine lines were treated using cosmetic products containing active agents acting on the skin, for example by moisturizing it or by improving its cell renewal or alternatively by promoting the synthesis of collagen, of which skin tissue is composed, or by preventing its degradation.

Although these treatments make it possible to act on the wrinkles and fine lines caused by chronological or intrinsic aging, and also on those caused by photoaging, they have no effect on expression wrinkles.

Specifically, expression wrinkles are the result of mechanisms different from those that generate the wrinkles caused by aging.

Also, specifically, they are produced due to the effect of the strain exerted on the skin by the skin muscles that allow facial expressions. Depending on the shape of the face, the frequency of facial expressions and possible tics, they may appear even from childhood. Age, and also certain environmental factors such as exposure to sunlight, do not play a part in generating them, but may make them deeper and permanent.

Microanatomical connections between the dermis and the underlying muscle have often been documented in the various areas of the face. As regards its anatomical connections, the continuous phenomena of contraction and relaxation produce tension forces in the connective tissue of the dermis. Over time, these tensile forces are thought to gradually modify the phenotype of the fibroblasts present in the area of the wrinkle, allowing them to acquire contractile properties and to induce remodeling of the dermal fibrous matrix. The consequence of this is an increased formation of expression lines that become permanent wrinkles.

Expression wrinkles are characterized by the presence of grooves around the orifices formed by the nose (nasal grooves), the mouth (perioral wrinkles and “sour-face” wrinkles) and the eyes (crow's-feet wrinkles), around which are the skin muscles, and also between the eyebrows (glabella wrinkles or lion wrinkles) and on the forehead.

Hitherto, the only means commonly used for acting on expression wrinkles is botulinum toxin, which is especially injected into the wrinkles of the glabella (see J. D. Carruters et al., J. Dermatol. Surg. Oncol., 1992, 18, pp. 17-21) and, moreover, degradable implants based on collagen, hyaluronic acid or polylactic acid.

In addition, as an alternative to these medical techniques requiring the intervention of a practitioner, the assignee hereof has proposed various compounds capable of affording a dermo-decontracting effect when they are applied topically to the skin, thus making it possible to act on expression wrinkles via another route. Among these compounds that may especially be mentioned are antagonists of the receptors associated with the calcium channels (FR-2,793,681), and in particular manganese and its salts (FR-2,809,005) and alverine (FR-2,798,590); and agonists of the receptors associated with the chlorine channels, including glycine (EP-0,704,210) and certain extracts of Iris pallida (FR-2,746,641).

However, need continues to exist for effective compounds for preventing and/or combating the signs of aging of the skin and for smoothing or fading out wrinkles, in particular expression wrinkles.

### SUMMARY OF THE INVENTION

It has now surprisingly and unexpectedly been determined that inhibition of the signaling pathway for the Rho kinases activated by the protein RhoA, also referred to as the RhoA kinase signaling pathway, satisfies the aforesaid need.

In particular, it has now been shown, on a model of dermal equivalent, that the use of a specific RhoA kinase inhibitor, (R)(+)-trans-N-(4-pyridyl)-4-(1-aminoethyl)cyclohexanecarboxamide dihydrochloride, also known as Y-27632, relaxes and/or decontracts the dermal contractile cells assumed to be involved in the generation of expression wrinkles. A similar effect has also been shown with another RhoA kinase inhibitor, 1-(5-isoquinolinesulfonyl)homopiperazine, which is also known as HA1077 or Fasudil. These inhibitors, which bind specifically to the catalytic site of the RhoA kinases, are inhibitors of activation of said RhoA kinases.

Decontraction of the cells in the dermis would thus prevent and/or combat the signs of aging of the skin, and, in particular, combat expression wrinkles.

RhoA kinase inhibitors have previously been described in the literature as having an inhibitory effect on smooth muscle contraction, suggesting administration as an anti-hypertensive agent in the therapeutic prevention and/or treatment of coronary, cerebral and renal diseases (WO 90/05723), the treatment of glaucoma (EP-1,034,793, WO 2000/057,914), asthma (JP 2000/063,274) or pulmonary fibrosis (EP-1,163,910).

However, the prior art does not at all suggest any effect of these RhoA kinase inhibitors on striated muscles, in particular on the skin muscles, or on the dermal contractile cells involved in the generation of expression wrinkles.

Now, it is known in the literature that compounds that are effective on smooth muscle do not necessarily have an effect on striated muscle, and vice-versa (American Journal of Veterinary Research (1996), 57(10), 1497-1500; Planta Medica (1970), 18(3), 222-6; Nippon Yakurigaku Zasshi (1976), 72(1), 41-52; Drug Development Research (1992), 25(2), 161-9).

Thus, it was not apparent that the inhibitors of RhoA kinase production and/or activation according to the present invention might have a relaxing effect on striated muscle, in particular on the skin muscles or on the dermal contractile cells involved in the generation of expression wrinkles; accordingly, their administration as dermo-decontracting agents in anti-aging cosmetic/dermatological compositions could not have been foreseen.

The present invention thus features the formulation into cosmetic compositions, of effective amounts of at least one inhibitor of RhoA kinase production and/or activation, as an agent for relaxing the features and/or for decontracting the skin.

The expression “inhibitor of RhoA kinase production and/or activation” especially means, according to the invention, any agent capable of inhibiting (i) the signaling pathways leading to the transcription or translation of the RhoA kinases, (ii) the processes of post-translational modification of the RhoA kinases, and/or (iii) the activation of said RhoA kinases.

### DETAILED DESCRIPTION OF BEST MODE AND SPECIFIC/PREFERRED EMBODIMENTS OF THE INVENTION

Preferably, the inhibitor is an inhibitor of activation of said RhoA kinases. This inhibitor of RhoA kinase activation may be selected from among (a) an inhibitor capable of binding to the ATP binding site, preventing the phosphorylation reaction of the said kinases, (b) an inhibitor capable of binding to the catalytic site of the said kinase, inducing a conformational change in which the kinase is inactive, (c) an inhibitor capable of binding to binding sites of its specific substrates, or (d) an inhibitor of activation by the protein RhoA of the said RhoA kinases, also known as an inhibitor of the RhoA-dependent activation of the said RhoA kinases.

Preferably, the inhibitor of activation of the said RhoA kinases is selected from an agent for binding to the catalytic site of the said RhoA kinases and an agent for inhibiting the RhoA-dependent activation of the said RhoA kinases.

According to a first embodiment of the invention, an inhibitor capable of binding to the catalytic site of the RhoA kinases is used.

Examples of such compounds that may be mentioned include the trans-4-amino(alkyl)-1-pyridylcarbamoylcyclohexane compounds described in U.S. Pat. No. 4,997,834 and the 4-amino(alkyl)cyclohexane-1-carboxamide compounds described in U.S. Pat. No. 5,478,838 and/or derivatives thereof and/or analogues thereof. Mention may also be made of 1-(5-isoquinolinesulfonyl)homopiperazine, also known as HA1077 or Fasudil sold by Calbiochem, and/or derivatives thereof and/or analogues thereof.

More preferably, the compound will be selected from (R)(+)-trans-4-(1-aminoethyl)-N-(4-pyridyl)cyclohexanecarboxamide, and/or derivatives thereof and/or analogues thereof.

The term “derivatives” especially means the salts, the substituted derivatives, the optical isomers and the racemic mixtures of the said compound.

Salts of the said compound that may be mentioned include the salts obtained by addition of the said compound to a mineral acid selected from among hydrochloric acid, sulfuric acid, nitric acid and phosphoric acid, or of an organic acid selected in particular from among malonic acid, succinic acid, fumaric acid, lactic acid, glycolic acid, citric acid and tartaric acid.

It will preferably be (R)(+)-trans-4-(1-aminoethyl)-N-(4-pyridyl)cyclohexanecarboxamide dihydrochloride (Y27632) sold by Calbiochem.

The term “analogues” especially means the enzymatic or biomimetic analogues of the said compound, capable of binding to the catalytic site of the RhoA kinases and thus of inhibiting their activation. Such analogues may be selected in vitro via tests of RhoA kinase binding or inactivation according to the standard techniques developed in enzymology and biochemistry.

The derivatives and/or analogues may be of natural or synthetic origin.

The term “natural origin” means a compound in pure form or as a solution at various concentrations, obtained via various extraction processes from a tissue (skin, etc.) of natural origin.

The term “synthetic origin” means a compound in pure form or as a solution at various concentrations, obtained chemically or enzymatically or by production in an organism after introduction into this organism of the components required for this production.

Synthetic analogues of the said compounds will preferably be used.

According to another embodiment of the invention, an inhibitor of the RhoA-dependent activation of the said RhoA kinases is used.

This inhibitor may be (a) an agent capable of inhibiting the production and/or activation of the protein RhoA, or (b) an inhibitor capable of preventing the interaction of the protein RhoA with its target, RhoA kinase, by binding, for example, to specific binding sites.

An example of an inhibitor of activation of the protein RhoA according to (a) that may be mentioned is an agent capable of preventing the conversion of the inactive form (GDP) of protein RhoA into its GTP-binding active form, such as an agent with ADP-ribosyltransferase activity leading to ribosylation of protein RhoA, which then becomes incapable of binding GTP.

Examples of agents with ADP-ribosyltransferase activity that may be mentioned include the enzyme C3 exotransferase, also known as C3, or an analogue.

The term “C3 exotransferase analogue” especially means any non-toxic agent or bacterial or cellular extract having C3 exotransferase activity. This C3 exotransferase activity may especially be purified from non-toxic strains of C and D type Clostridium botulinum, which synthesize it. This C3 activity is moreover only found in these C and D types, and is not produced by the neurotoxic strains of A, B and E type (Rubin, E. J. et al., Molecular and Cellular Biology, January 1988, Vol. 8, No. 1, pp. 418-426).

The derivatives and/or analogues of the compounds Y27632, HA1077 or C3 exotransferase and more generally of the inhibitors of RhoA kinase activation, which are suitable for use in the invention, may be:
prepared via chemical or enzymatic synthesis or via combinatorial chemistry and tested for their capacity to bind to and/or to inactivate, respectively, RhoA kinase or the protein RhoA, according to the standard techniques developed in enzymology and biochemistry;
selected for their dermo-decontracting activity, for example on a dermal equivalent.

To evaluate the dermo-decontracting activity of the inhibitors according to the invention, a test comprising the following steps will preferably be employed:
a dermal equivalent is prepared, on a support, in a suitable culture medium comprising at least one inhibitor according to the invention to be tested;
the spontaneous contraction of the dermal equivalents is measured and the spontaneous contraction of the dermal equivalent prepared with the said inhibitor is compared with that of a dermal equivalent prepared without the said inhibitor (control dermal equivalent);
the compounds for which the spontaneous contraction of the dermal equivalent in the presence of the said inhibitor is reduced by at least 3% and preferably by at least 8% relative to the spontaneous contraction of the control dermal equivalent are selected.

The term “support” means any culture support suitable for preparing the dermal equivalent. An example that may be mentioned is a 12-well culture plate (such as Costar reference 3512).

The term “dermal equivalent” means, for example, any collagen matrix seeded with skin cells, selected at least from fibroblasts and myofibroblasts obtained via in vitro differentiation, on a culture support.

Examples of preparation of dermal equivalents that may be mentioned include the protocols described in EP-A-285,471, EP-A-285,474, EP-A-789,074, EP-A-502,172, EP-A-418,035, WO-A-91/16010, EP-A-197,090, EP-A-20,753, FR-A-2,665,175, FR-A-2,689,904 or, preferably, the protocol described by Asselineau et al., 1987, (Models in Dermato., Vol. III, Ed. Lower & Maibach, 1-7).

Alternatively, it is possible to use:
either a free dermal equivalent: once formed, the dermal equivalent is immediately detached from the culture support and its contraction starts;
or an attached dermal equivalent: once formed, the dermal equivalent is left adhering to the culture support for a certain time, and then detached from the support in order for the contraction to be able to start.

The term “suitable culture medium” means a nutrient culture medium that comprises at least the components required for the growth and survival of the skin cells, especially fibroblasts or myofibroblasts.

As a culture medium that is well known to those skilled in the art, an example that may be mentioned is the MEM medium (minimum essential medium).

The inhibitory compounds to be tested are used at concentrations of between 10⁻⁸ M and 10⁻³ M and preferably between 10⁻⁷ M and 10⁻⁵ M.

The term “spontaneous contraction” of the dermal equivalent means the effect of the intercellular retractions establishing tensile forces between the cells and their surrounding medium. These forces induce a reduction in the area of the dermal equivalent over time, which it is possible to measure, especially by image analysis. From these area measurements, a percentage of contraction is deduced, which makes it possible to assess the in vitro “microtension” phenomenon.

The measurement of spontaneous contraction of the dermal equivalents entails measuring the area of the dermal equivalents and in deducing therefrom a corresponding level of spontaneous contraction. It may be performed via any system known to those skilled in the art.

Preferably, a digital imaging system combined with image analysis software will be used. In particular, the digital image is acquired using a Sony DXC-107P CDD-Iris camera and is then transcribed into an area measurement and percentage of contraction by means of a Zeiss Axiovision 3.0 image analysis system.

The said inhibitor of RhoA kinase production and/or activation according to the invention is especially intended for relaxing and/or decontracting the contractile cells of the dermis, in particular the contractile fibroblasts.

The present invention also features formulating into cosmetic compositions, of at least one inhibitor of RhoA kinase production and/or activation, the said inhibitor being suited to smooth out the skin and/or to attenuate or efface the skin microrelief.

The skin microrelief according to the invention is defined by microdepressions at the surface of the skin generated by the contraction of the skin cells, in particular of the cells of the dermis.

The said inhibitor of RhoA kinase production and/or activation is also suited to reduce and/or smooth out wrinkles.

In particular, the said inhibitor is suited to reduce and/or smooth out expression wrinkles.

The invention also features formulating into cosmetic compositions, of at least one inhibitor of RhoA kinase production and/or activation, the said composition being suitable for oral administration or for topical application, preferably for topical application.

A formulation suitable for the oral route may be in the form of coated tablets, gel capsules, gels, emulsions, tablets, capsules or liquid solutions, especially drinkable vials, for example. In particular, the active agent(s) according to the invention may be incorporated into all other forms of dietary supplements or of enriched foods, for example dietary bars, or compacted or non-compacted powders.

This invention also features cosmetic compositions for topical application, comprising, in a physiologically acceptable medium, an effective amount of at least one inhibitor of RhoA kinase production and/or activation as defined above.

According to the invention, the term “physiologically acceptable medium” means a medium that is compatible with the skin and possibly its integuments (eyelashes, nails or hair) and/or mucous membranes.

In particular, the composition comprises an effective amount of at least one inhibitor of RhoA kinase activation selected from (R)(+)-trans-4-(1-aminoethyl)-N-(4-pyridyl)cyclohexanecarboxamide and 1-(5-isoquinolinesulfonyl)homopiperazine, derivatives thereof or analogues thereof.

The amount of the said inhibitor of RhoA kinase production and/or activation obviously depends on the desired effect and may thus vary within a wide range.

To provide an order of magnitude, the said inhibitor may be present in the composition in an amount from 0.0000001% to 10%, preferably from 0.000001% to 1% relative to the total weight of the composition, and even more preferably from 0.00001% to 0.1% relative to the total weight of the composition.

The compositions according to the invention may be in any galenical form normally used in cosmetics, which is suitable for the topical route.

The composition may especially be in the form of an optionally gelled aqueous solution, a dispersion of the lotion type, which is optionally a two-phase dispersion, an emulsion obtained by dispersing a fatty phase in an aqueous phase (O/W) or conversely (W/O), or a triple emulsion (W/O/W or O/W/O) or a vesicular dispersion of ionic and/or nonionic type. These compositions are prepared according to the usual methods.

It may have the appearance of a white or colored cream, an ointment, a milk, a lotion, a gel, a serum, a paste or a mousse, for example.

It may also be in solid form, in particular in the form of a stick.

It may also be used as a care product and/or as a makeup product for the skin.

In a known manner, the compositions according to the invention may also contain adjuvants that are common in cosmetics, such as hydrophilic or lipophilic gelling agents, hydrophilic or lipophilic active agents, preservatives, antioxidants, solvents, fragrances, fillers, screening agents, pigments, odor absorbers and dyestuffs. The amounts of these various adjuvants are those conventionally used in the field under consideration, and, for example, from 0.01% to 20% relative to the total weight of the composition. Depending on their nature, these adjuvants may be introduced into the fatty phase, into the aqueous phase, or into lipid vesicles. In any case, these adjuvants, and also the proportions thereof, will be selected so as not to harm the desired properties of the inhibitor of RhoA kinase activation.

When the compositions according to the invention are emulsions, the proportion of the fatty phase may range from 5% to 80% by weight and preferably from 5% to 50% by weight relative to the total weight of the composition. The oils, emulsifiers and co-emulsifiers used in the composition in emulsion form are selected from those conventionally used in the field under consideration. The emulsifier and co-emulsifier are present in the composition in a proportion ranging from 0.3% to 30% by weight and preferably from 0.5% to 20% by weight relative to the total weight of the composition.

As oils that may be used in the invention, mention may be made of hydrocarbons of mineral or synthetic origin (liquid petroleum jelly, isohexadecane), oils of plant origin (apricot kernel oil, liquid fraction of shea butter, avocado oil or soybean oil), oils of animal origin (lanolin), synthetic oils (perhydrosqualene, pentaerythrityl tetraoctanoate), silicone oils (cyclopentasiloxane and cyclohexasiloxane) and fluoro oils (perfluoropolyethers). Fatty alcohols (cetyl alcohol or stearyl alcohol), fatty acids (stearic acid) and waxes (carnauba wax, ozokerite or beeswax) may also be used as fatty substances.

As examples of emulsifiers and co-emulsifiers that may be used in the invention, mention may be made of fatty acid esters of polyethylene glycol such as PEG-100 stearate and PEG-20 stearate, and fatty acid esters of glycerol such as glyceryl stearate.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

Preservatives that may be mentioned include para-hydroxybenzoic acid esters, 1,2-octanediol, 3-iodo-2-propynyl butyl carbamate, phenoxyethanol and chlorhexidine gluconate.

Examples of fillers that may be mentioned include polyamide (Nylon) particles; polymethyl methacrylate microspheres; ethylene-acrylate copolymer powders; expanded powders such as hollow microspheres and especially microspheres formed from a terpolymer of vinylidene chloride, of acrylonitrile and of methacrylate, and sold under the name Expancel by Kemanord Plast; powders of natural organic materials such as starch powders, especially corn, wheat or rice starch, which may or may not be crosslinked, such as powders of starch crosslinked with octenylsuccinate anhydride; silicone resin microbeads such as those sold under the name Tospearl by Toshiba Silicone; silica; metal oxides such as titanium dioxide or zinc oxide; mica; and mixtures thereof.

As hydrophilic or lipophilic active agents, it will be advantageous to introduce into the composition according to the invention at least one compound selected from: other dermo-decontracting agents; moisturizers; depigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; tensioning agents; antipollution agents and/or free-radical scavengers; UV-screening agents; and mixtures thereof.

Examples of such additional compounds are given below.

As dermo-decontracting agents other than the inhibitors of RhoA kinase activation according to the invention, mention may be made of: antagonists of receptors associated with the calcium channels (FR-2,793,681), in particular manganese and its salts (FR-2,809,005) and alverine and its salts (FR-2,798,590), especially alverine citrate; agonists of receptors associated with the chlorine channels, including glycine (EP-0,704,210) and certain extracts of Iris pallida (FR-2,746,641); sapogenins such as diosgenin and natural extracts containing them (such as extracts of wild yam), certain secondary and tertiary carbonyl amines, organic or mineral salts of metals, in particular manganese gluconate, adenosine, and also argireline R hexapeptide sold by Lipotec. Mention may also be made of extract of Boswellia serrata and certain fragrancing compositions with a dermo-decontracting effect.

The term “moisturizer” means:
either a compound acting on the barrier function, in order to keep the stratum corneum moisturized, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;
or a compound that directly increases the water content of the stratum corneum, such as trehalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroylpyrrolidonecarboxylic acid and N-α-benzoyl-L-arginine;
or a compound that activates the sebaceous glands, such as DHEA, the 7-oxido and/or 17-alkyl derivatives thereof, sapogenins, and vitamin D and its derivatives.

The depigmenting agents that may be incorporated into the compositions according to the present invention comprise, for example, the following compounds: kojic acid; ellagic acid; arbutin and its derivatives such as those described in patent applications EP-895,779 and EP-524,109; hydroquinone; aminophenol derivatives such as those described in WO 99/10318 and WO 99/32077, and in particular N-cholesteryloxycarbonyl-para-aminophenol and N-ethyloxycarbonyl-para-aminophenol; iminophenol derivatives, in particular those described in WO 99/22707; L-2-oxothiazolidine-4-carboxylic acid or procysteine, and also its salts and esters; ascorbic acid and its derivatives, especially ascorbyl glucoside; and plant extracts, in particular extracts of liquorice, of mulberry and of skullcap, without this list being limiting.

The term “anti-glycation agent” means a compound for preventing and/or reducing the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetrahydroxystilbene.

Examples of NO-synthase inhibitors that are suitable for use in the present invention especially comprise a plant extract of the species Vitis vinifera which is sold especially by Euromed under the name “Leucocyanidines de raisins extra”, or by Indena under the name Leucoselect®, or finally by Hansen under the name “Extrait de marc de raisin”; a plant extract of the species Olea europaea which is preferably obtained from olive tree leaves and is sold especially by Vinyals in the form of a dry extract, or by Biologia & Technologia under the trademark Eurol BT; and a plant extract of the species Gingko biloba which is preferably a dry aqueous extract of this plant sold by Beaufour under the trademark “Gingko biloba extrait standard”.

Among the active agents for stimulating dermal macromolecules or for preventing their degradation, mention may be made of those that act:
either on collagen synthesis, such as extracts of Centella asiatica; asiaticosides and derivatives; ascorbic acid or vitamin C and its derivatives; synthetic peptides such as iamin, biopeptide CL or palmitoyloligopeptide sold by Sederma; peptides extracted from plants, such as the soybean hydrolysate sold by Coletica under the trademark Phytokine®; and plant hormones such as auxins and lignans;
or on elastin synthesis, such as the extract of Saccharomyces cerivisiae sold by LSN under the trademark Cytovitin®; and the extract of the alga Macrocystis pyrifera sold by SECMA under the trademark Kelpadelie®;
or on glycosaminoglycan synthesis, such as the product of fermentation of milk with Lactobacillus vulgaris, sold by Brooks under the trademark Biomin yogourth®; the extract of the brown alga Padina pavonica sold by Alban Müller under the trademark HSP3®; and the extract of Saccharomyces cerevisiae available especially from the company Silab under the trademark Firmalift® or from the company LSN under the trademark Cytovitin®;
or on fibronectin synthesis, such as the extract of the zooplankton Salina sold by Seporga under the trademark GP4G®; the yeast extract available especially from the company Alban Müller under the trademark Drieline®; and the palmitoyl pentapeptide sold by Sederma under the trademark Matrixil®;
or on the inhibition of metalloproteases (MMP), such as, more particularly, MMP 1, 2, 3 or 9. Mention may be made of: retinoids and derivatives, oligopeptides and lipopeptides, lipoamino acids, the malt extract sold by Coletica under the trademark Collalift®; extracts of blueberry or of rosemary; lycopene; isoflavones, their derivatives or plant extracts containing them, in particular extracts of soybean (sold, for example, by Ichimaru Pharcos under the trademark Flavosterone SB®), of red clover, of flax, of kakkon, or of sage;
or on the inhibition of serine proteases such as leukocyte elastase or cathepsin G. Mention may be made of: the peptide extract of Leguminosa seeds (Pisum sativum) sold by LSN under the trademark Parelastyl®; and heparinoids and pseudodipeptides such as {2-[acetyl(3-trifluoromethylphenyl)amino]-3-methylbutyrylamino}acetic acid.

Among the active agents that stimulate epidermal macromolecules, such as fillagrin and keratins, mention may be made especially of the extract of lupin sold by Silab under the trademark Structurine®; the extract of beech Fagus sylvatica buds sold by Gattefosse under the trademark Gatuline®; and the extract of the zooplankton Salina sold by Seporga under the trademark GP4G®.

The agents for stimulating fibroblast proliferation that may be used in the composition according to the invention may be selected, for example, from plant proteins or polypeptides, extracted especially from soybean (for example an extract of soybean sold by LSN under the name Eleseryl SH-VEG 8® or sold by Silab under the trademark Raffermine®); and plant hormones such as giberrellins and cytokinins.

The agents for stimulating keratinocyte proliferation that may be included in the compositions according to the invention especially comprise retinoids such as retinol and its esters, including retinyl palmitate; phloroglucinol; extracts of walnut cakes sold by Gattefosse; and extracts of Solanum tuberosum sold by Sederma.

The agents for stimulating keratinocyte differentiation comprise, for example, minerals such as calcium; the extract of lupin sold by Silab under the trademark Photopreventine®; sodium β-sitosteryl sulfate sold by Seporga under the trademark Phytocohesine®; and the extract of corn sold by Solabia under the trademark Phytovityl®; and lignans such as secoisolariciresinol.

The term “tensioning agent” means a compound capable of exerting tension on the skin, the effect of which is to temporarily fade out irregularities on the skin's surface, such as wrinkles and fine lines.

Among the tensioning agents that may be used in the composition according to the present invention, mention may be made especially of:
(1) synthetic polymers such as polyurethane latices or acrylic-silicone latices, in particular those described in EP-1,038,519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold especially by 3M under the trademarks VS 80, VS 70 or LO21,
(2) polymers of natural origin, especially (a) polyholosides, for example (i) in the form of starch derived especially from rice, corn, potato, cassaya, pea, Triticum aestivum wheat, oat, etc. or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins and cellulose-based derivatives, and mixtures thereof,
(3) plant proteins and protein hydrolysates, in particular from corn, rye, Triticum aestivum wheat, buckwheat, sesame, spelt, pea, bean, lentil, soybean and lupin,
(4) mixed silicates, especially phyllosilicates and in particular Laponites,
(5) wax microparticles chosen, for example, from carnauba wax, candelilla wax and esparto grass wax,
(6) colloidal particles of mineral filler with a number-average diameter of from 0.1 to 100 nm and preferably from 3 to 30 nm, selected, for example, from: silica, silica-alumina composites, cerium oxide, zirconium oxide, alumina, calcium carbonate, barium sulfate, calcium sulfate, zinc oxide and titanium dioxide.

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be included in the compositions according to the invention, mention may be made in particular of vitamin C and its derivatives including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolysed RNA, sold by Laboratoires Serobiologiques under the trademark CPP LS 2633-12F®, the water-soluble fraction of corn sold by Solabia under the trademark Phytovityl®, the mixture of extract of fumitory and of extract of lemon sold under the name Unicotrozon C-49® by Induchem, and the mixture of extracts of ginseng, of apple, of peach, of wheat and of barley, sold by Provital under the trademark Pronalen Bioprotect®.

As agents for trapping monocyclic or polycyclic aromatic compounds, which may be included in the compositions according to the invention, mention may be made in particular of tannins such as ellagic acid; indole derivatives, in particular 3-indolecarbinol; extracts of tea, in particular of green tea, extracts of water hyacinth or Eichhomia crassipes; and the water-soluble fraction of corn sold by Solabia under the trademark Phytovityl®.

Finally, as heavy-metal-trapping agents that may be included in the compositions according to the invention, mention may be made in particular of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichhomia crassipes); and the water-soluble fraction of corn sold by Solabia under the trademark Phytovityl®.

The free-radical scavengers that may be included in the compositions according to the invention comprise, besides certain anti-pollution agents mentioned above, vitamin E and its derivatives such as tocopheryl acetate; bioflavonoids; coenzyme Q10 or ubiquinone; certain enzymes, for instance catalase, superoxide dismutase, lactoperoxidase, glutathione peroxidase and quinone reductases; glutathione; benzylidenecamphor; benzylcyclanones; substituted naphthalenones; pidolates; phytanetriol; gamma-oryzanol; lignans; and melatonin.

As indicated previously, the compositions according to the invention may also contain UV-A and/or UV-B screening agents, in the form of organic or mineral compounds, the latter being optionally coated to make them hydrophobic.

The organic photoprotective agents that are more particularly preferred are selected from among the following compounds: ethylhexyl salicylate, ethylhexyl methoxycinnamate, octocrylene, phenylbenzimidazole sulfonic acid, benzophenone-3, benzophenone-4, benzophenone-5, 4-methylbenzylidenecamphor, terephthalylidenedicamphorsulfonic acid, disodium phenyl dibenzimidazole tetrasulfonate, 2,4,6-tris(diisobutyl 4′-aminobenzalmalonate)-s-triazine, anisotriazine, ethylhexyl triazone, diethylhexylbutamido triazone, methylenebisbenzotriazolyl tetramethylbutylphenol, drometrizole trisiloxane, 1,1-dicarboxy(2,2′-dimethylpropyl)-4,4-diphenylbutadiene, and mixtures thereof.

The mineral photoprotective agents are selected from pigments or nanopigments (mean size of the primary particles: generally from 5 nm to 100 nm and preferably from 10 nm to 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are described in particular in EP-518,772 and EP-518,773.

The photoprotective agents are generally present in the compositions according to the invention in proportions ranging from 0.1% to 20% by weight relative to the total weight of the composition, and preferably ranging from 0.2% to 15% by weight relative to the total weight of the composition.

The present invention also features a cosmetic regime or regimen for reducing wrinkles and/or for relaxing the features and/or for decontracting the skin, comprising the topical application to the skin of a composition containing, in a physiologically acceptable medium, an effective amount of at least one inhibitor of RhoA kinase production and/or activation as defined above.

This process is suitable for treating wrinkled and/or aged skin and is directed especially towards preventing and/or reducing expression wrinkles.

In particular, the composition may be applied to the areas of the face or forehead marked with expression wrinkles and/or to individuals who have expression wrinkles.

The wrinkles to be treated are selected especially from the wrinkles lying radially around the mouth and/or the eyes and/or horizontally on the forehead and/or located in the space between the eyebrows.

The composition may be applied daily to the areas of the face or forehead marked with expression wrinkles.

In order to further illustrate the present invention and the advantages thereof, the following specific examples are given, it being understood that same are intended only as illustrative and in nowise limitative. In said examples to follow, all parts and percentages are given by weight, unless otherwise indicated.

In these examples, reference is made to the attached figure, which illustrates the level of contraction over time of a dermal equivalent treated with (R)(+)-trans-N-(4-pyridyl)-4-(1-aminoethyl)cyclohexanecarboxamide dihydrochloride.

### EXAMPLE 1

### Demonstration of the Dermo-Decontracting Effect of (R)(+)-trans-N-(4-pyridyl)-4-(1-aminoethyl)cyclohexanecarboxamide dihydrochloride or Y27632

a) Principle of the Test:

The principle of this test entails studying the dermo-decontracting effect of (R)(+)-trans-N-(4-pyridyl)-4-(1-aminoethyl)cyclohexanecarboxamide dihydrochloride (Y-27632) on a model of dermal equivalent comprising a collagen matrix seeded with normal human fibroblasts.

These conditions are intended to mimic in vitro the dermal contractile phenomena that take place during facial expressions. Under these conditions, specifically, the cells spontaneously express tensile forces that induce a retraction of the collagen gel. This results in a reduction of the total surface area of the dermal equivalent over time. Measurement of this area makes it possible to evaluate the relaxation effects of substances placed in contact beforehand with the dermal equivalent.

b) Protocol:

Four series of 3 attached dermal equivalents containing normal human fibroblasts are prepared: a control series without any treatment, and three series treated with (R)(+)-trans-N-(4-pyridyl)-4-(1-aminoethyl)cyclohexanecarboxamide dihydrochloride at different concentrations. The experiment is repeated three times.

The dermal equivalents are prepared as described in Asselineau et al., Exp. Cell. Res. 1985, 159, 536-539; Models in Dermatology, 1987, Vol. 33, pp. 1-7, in the following proportions:

| | |
|---|---|
| MEM medium (1.76X) with or without Y-27632 | 45% |
| Foetal calf serum: | 9% |
| NaOH (0.1 N): | 5% |
| Acetic acid (1/1000): | 4% |
| Collagen: | 26% |
| Fibroblasts: | 11% |

The treated dermal equivalent differs from the control dermal equivalent in that variable amounts of (R)(+)-trans-N-(4-pyridyl)-4-(1-aminoethyl)cyclohexanecarboxamide dihydrochloride are added thereto.

The collagen used is type I collagen (commercial solution), but a collagen of type III or IV may also be used. It is extracted from rat tails or from calf skin by acidic hydrolysis and stored in acidic medium at +4° C.; it naturally polymerizes on heating to 37° C. and on reducing the level of acidity. The collagen is predialyzed against successive baths of water+acetic acid.

The protocol is as follows: 1.76×MEM medium in the presence of additives (1% glutamine, 1% nonessential amino acids, 1% sodium pyruvate, 1% fungizone and 1% penicillin/streptomycin), foetal calf serum and 0.1 N NaOH are introduced into a sterile flask tube. The fibroblasts isolated from human skin explants are then added at a concentration of 1.4×10⁵ cells per 1 ml of culture medium.

A volume/volume mixture of collagen in acetic acid to 1/1000 is then added slowly, against the wall of the tube so as to observe the appearance of a whitish cloud.

The whole is then mixed cautiously and distributed in the wells of a 12-well culture plate (of the type Costar reference 3512) at a rate of 0.5 ml of mixture per cm². The culture plate is then placed in an incubator at 37° C. with 5% CO₂.

Once formed after polymerization of the collagen, the dermal equivalents are left adhering to the culture support for 3 days and then detached from the support in order for the contraction to be able to start. These attached dermal equivalents are removed from the incubator for the taking of the images to measure their surface area, for each point of the contraction kinetics (0, 4, 8 and 24 hours). They are immediately returned to the incubator between each measuring point.

The evaluation of the spontaneous contraction of the treated dermal equivalents (treated with (R)(+)-trans-N-(4-pyridyl)-4-(1-aminoethyl)cyclohexanecarboxamide dihydrochloride) and control dermal equivalents (without (R)(+)-trans-N-(4-pyridyl)-4-(1-aminoethyl)cyclohexanecarboxamide) is performed by measuring their surface area, at different times after the start of the spontaneous contraction.

To do this, a digital image is acquired for each treated or untreated dermal equivalent using a camera (Sony DXC-107P CCD-Iris camera) and the area is then calculated on each image using an image analysis system (Zeiss Axiovision 3.0). This area measurement corresponds to a percentage of contraction equal to the ratio of the areas, according to the formula:
% contraction=(Sw−Si)/Sw×100
in which:
“Sw” represents the area of a well of the culture plate; it corresponds to the total area of the dermal equivalent before contraction;
“Si” represents the area of the dermal equivalent at the time i of the contraction kinetics.

c) Results:

As illustrated in the attached Figure of Drawing, the level of contraction of the control dermal equivalent is 30.5% four hours after detaching it from its support. It rises to 35.5% after 8 hours and reaches 44.2% after 24 hours.

In this model, (R)(+)-trans-N-(4-pyridyl)-4-(1-aminoethyl)cyclohexanecarboxamide dihydrochloride induces a dose-dependent inhibition of contraction of the dermal equivalent, which is, moreover, reversible.

At a concentration of 0.1 μM, (R)(+)-trans-N-(4-pyridyl)-4-(1-aminoethyl)cyclohexanecarboxamide dihydrochloride reduces this percentage of contraction by 6.1% after four hours, 6.4% after 8 hours and 7.2% after 24 hours, relative to the control.

Furthermore, at a concentration of 1 μM, (R)(+)-trans-N-(4-pyridyl)-4-(1-aminoethyl)cyclohexanecarboxamide dihydrochloride reduces this percentage of contraction by 20.6% after four hours, 24.4% after 8 hours and 29.3% after 24 hours, relative to the control.

This test thus demonstrates that (R)(+)-trans-N-(4-pyridyl)₄-(1-aminoethyl)cyclohexanecarboxamide results in reduced contraction of the dermal equivalent, and thus a relaxing or dermo-decontracting effect that may be exploited in the preparation of anti-aging cosmetic compositions, in particular for reducing and/or smoothing out wrinkles, and more particularly expression wrinkles.

### EXAMPLE 2

### Demonstration of the Dermo-Decontracting Effect of 1-(5-isoquinolinesulfonyl)homopiperazine or Fasudil (HA1077)

1-(5-Isoquinolinesulfonyl)homopiperazine was tested at different concentrations according to the protocol described in Example 1.

The results obtained are as follows:

The level of contraction of the control dermal equivalent is 30.5% four hours after detaching it from its support. It rises to 35.5% after 8 hours and reaches 44.2% after 24 hours.

In this model, 1-(5-isoquinolinesulfonyl)homopiperazine induces a dose-dependent inhibition of contraction of the dermal equivalent.

At a concentration of 0.1 μM, 1-(5-isoquinolinesulfonyl)homopiperazine reduces this percentage of contraction by 2.6% after 4 hours, 2.4% after 8 hours and 1.5% after 24 hours, relative to the control.

At a concentration of 1 μM, 1-(5-isoquinolinesulfonyl)homopiperazine reduces this percentage of contraction by 8% after four hours, 9.5% after eight hours and 8.2% after twenty-four hours, relative to the control.

At a concentration of 5 μM, 1-(5-isoquinolinesulfonyl)homopiperazine reduces this percentage of contraction by 17% after four hours, 19.4% after eight hours and 22.2% after twenty-four hours, relative to the control.

This test thus demonstrates that 1-(5-isoquinolinesulfonyl)homopiperazine, although less effective than (R)(+)-trans-N-(4-pyridyl)-4-(1-aminoethyl)cyclohexanecarboxamide dihydrochloride at the same concentrations, also gives rise to a lesser contraction of the dermal equivalent, and thus a relaxing or dermo-decontracting effect that may be exploited in the preparation of anti-aging cosmetic compositions, in particular for reducing and/or smoothing out wrinkles, and more particularly expression wrinkles.

### EXAMPLE 3

### Cosmetic Composition

This composition is prepared in a conventional manner:

Cream:

| | |
|---|---|
| (R)(+)-trans-N-(4-pyridyl)-4-(1-aminoethyl) | 0.001% |
| cyclohexanecarboxamide | |
| Stearic acid | 3.00% |
| Mixture of glyceryl monostearate and of | 2.50% |
| polyethylene glycol stearate (100 EO) | |
| Polyethylene glycol stearate (20 EO) | 1.00% |
| Cyclopentadimethylsiloxane | 10.00% |
| Fillers | 3.00% |
| Plant oils | 7.00% |
| Synthetic oils | 6.00% |
| Preservatives | 1.20% |
| Oxyethylenated (16 EO) dimethylsiloxane | 1.00% |
| containing methoxy end groups | |
| Silicone gum | 0.20% |
| Acrylic copolymer as a reverse emulsion | 1.70% |
| (Simulgel 600 from SEPPIC) | |
| Stearyl alcohol | 1.00% |
| Water | qs 100% |

This cream is applied to the face and forehead to attenuate expression wrinkles and to decontract the face.

Similar compositions in which the (R)(+)-trans-N-(4-pyridyl)-4-(1-aminoethyl)cyclohexanecarboxamide is replaced with 1-(5-isoquinolinesulfonyl)homopiperazine may also be prepared.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A regime or regimen for relaxing the features of and/or for decontracting the skin, comprising administering to an individual in need of such treatment, for such period of time as required to elicit the desired effect, a thus effective amount of at least one inhibitor of RhoA kinase production and/or activation, formulated into a physiologically acceptable medium therefor.

2. The regime or regimen as defined by claim 1, said at least one inhibitor of RhoA kinase production and/or activation binding to the catalytic site of said RhoA kinases.

3. The regime or regimen as defined by claim 1, said at least one inhibitor of RhoA kinase production and/or activation comprising (R)(+)-trans-4-(1-aminoethyl)-N-(4-pyridyl)cyclohexanecarboxamide, 1-(5-isoquinolinesulfonyl)homopiperazine and/or derivative and/or analogue thereof.

4. The regime or regimen as defined by claim 1, said at least one inhibitor being of RhoA kinase activation.

5. The regime or regimen as defined by claim 4, said at least one inhibitor of RhoA kinase activation comprising an agent for inhibiting the activation, by the protein RhoA, of RhoA kinases.

6. The regime or regimen as defined by claim 5, said activation inhibiting agent exhibiting ADP-ribosyltransferase activity.

7. The regime or regimen as defined by claim 6, said agent exhibiting ADP-ribosyltransferase activity comprising C3 exotransferase and/or analogue thereof.

8. A regime or regimen for smoothing the skin and/or attenuating the microrelief thereof, comprising administering to an individual in need of such treatment, for such period of time as required to elicit the desired effect, a thus effective amount of at least one inhibitor of RhoA kinase production and/or activation, formulated into a physiologically acceptable medium therefor.

9. A regime or regimen for reducing and/or smoothing skin wrinkles, comprising administering to an individual in need of such treatment, for such period of time as required to elicit the desired effect, a thus effective amount of at least one inhibitor of RhoA kinase production and/or activation, formulated into a physiologically acceptable medium therefor.

10. The regime or regimen as defined by claim 9, said skin wrinkles comprising expression wrinkles.

11. The regime or regimen as defined by claim 1, comprising topically applying said at least one inhibitor of RhoA kinase production and/or activation and physiologically acceptable medium therefor onto the affected skin area of said individual in need of such treatment.

12. The regime or regimen as defined by claim 11, comprising topically applying said at least one inhibitor of RhoA kinase production and/or activation and physiologically acceptable medium therefor onto that skin area of the face and/or forehead marked with expression wrinkles.

13. The regime or regimen as defined by claim 1, comprising orally administering said at least one inhibitor of RhoA kinase production and/or activation and physiologically acceptable medium therefor to said individual in need of such treatment.

14. A topically applicable cosmetic/dermatological composition suited for relaxing the features of and/or for decontracting the skin, comprising a thus effective amount of at least one inhibitor of RhoA kinase production and/or activation which comprises (R)(+)-trans-4-(1-aminoethyl)-N-(4-pyridyl)cyclohexanecarboxamide, 1-(5-isoquinolinesulfonyl)homopiperazine or derivative or analogue thereof, together with at least one adjuvant selected from the group consisting of antioxidants, fragrances, fillers, UV-screening agents, pigments, odor absorbers and dyestuffs, formulated into a topically applicable, physiologically acceptable medium therefor.

15. The cosmetic/dermatological composition as defined by claim 14, said at least one inhibitor of RhoA kinase production and/or activation comprising from 0.0000001% to 10% by weight thereof.

16. The cosmetic/dermatological composition as defined by claim 14, said at least one inhibitor of RhoA kinase production and/or activation comprising from 0.000001% to 1% by weight thereof.

17. The cosmetic/dermatological composition as defined by claim 14, said at least one inhibitor of RhoA kinase production and/or activation comprising from 0.00001% to 0.1% by weight thereof.

18. The cosmetic/dermatological composition as defined by claim 14, further comprising at least one hydrophilic or lipophilic active agent selected from the group consisting of other dermo-decontracting agents, moisturizers, depigmenting agents, anti-glycation agents, NO-synthase inhibitors, agents for stimulating the synthesis of dermal or epidermal macromolecules and/or for preventing degradation thereof, agents for stimulating fibroblast and/or keratinocyte proliferation or for stimulating keratinocyte differentiation, tensioning agents, anti-pollution agents and/or free-radical scavengers, and mixtures thereof.

19. Methodology for determining the identity of an inhibitor of RhoA kinase activation, comprising the following steps:
placing a dermal equivalent, on a support, in a suitable culture medium which comprises at least one inhibitor of RhoA kinase activation sought to be identified;
measuring the spontaneous contraction of a dermal equivalent treated with the said inhibitor and comparing same with the spontaneous contraction of a control dermal equivalent untreated with said inhibitor;
identifying said inhibitors for which the spontaneous contraction of the dermal equivalent in the presence of inhibitor is reduced by at least 3% relative to the spontaneous contraction of the dermal equivalent without inhibitor.

20. Methodology for determining the identity of an inhibitor of RhoA kinase activation, comprising the following steps:
placing a dermal equivalent, on a support, in a suitable culture medium which comprises at least one inhibitor of RhoA kinase activation sought to be identified;
measuring the spontaneous contraction of a dermal equivalent treated with the said inhibitor and comparing same with the spontaneous contraction of a control dermal equivalent untreated with said inhibitor;
identifying said inhibitors for which the spontaneous contraction of the dermal equivalent in the presence of inhibitor is reduced by at least 8% relative to the spontaneous contraction of the dermal equivalent without inhibitor.

21. The cosmetic/dermatological composition as defined by claim 1, formulated as an optionally gelled aqueous solution, cream, lotion, dispersion, emulsion, vesicular dispersion, gel, serum, paste, mousse, stick, or makeup.
